(19) United States
(12) Patent Application Publication
(10) Pub. No.: US 2026/0294415 A1
(43) Pub. Date: Oct. 1, 2026

(54) LEFT ATRIAL APPENDAGE OCCLUSION ASSEMBLIES

(71) Applicants: Medtronic, Inc., Minneapolis, MN (US); Covidien LP, Mansfield, MA (US); Medtronic CryoCath LP, Toronto (CA)

(72) Inventors: Junwei Li, Irvine, CA (US); Robert A. Pecor, Aliso Viejo, CA (US); Kevin R. Seifert, Forest Lake, MN (US); Thomas A. Anderson, New Hope, MN (US); Brian T. Howard, Minneapolis, MN (US); Bushan K. Purushothaman, Santa Rosa, CA (US); Nicolas Coulombe, Anjou (CA); Randal C. Schulhauser, Phoenix, AZ (US); Zhongping Yang, Woodbury, MN (US)

(21) Appl. No.: 19/479,231

(22) PCT Filed: Apr. 24, 2024
(86) PCT No.: PCT/US2024/025992

§ 371 Date: Oct. 27, 2025

Related U.S. Application Data
(60) Provisional application No. 63/499,014, filed on Apr. 28, 2023.

(51) Int. Cl. A61B 17/00

(52) U.S. Cl. CPC A61B 17/0057; A61B 17/00234; A61B 2017/00243; A61B 2017/00292; A61B 2017/00557; A61B 2017/00592; A61B 2017/00623; A61B 2017/00632; A61B 2017/00659; A61B 2017/00862

(57) ABSTRACT

In some examples, an example assembly for occluding an anatomical chamber includes an occluding pouch and a plug. The occluding pouch is configured to expand by receiving a volume of biogel to occlude the anatomical chamber. The plug includes at least one retention member configured to secure the occluding pouch to a wall of the anatomical chamber. In some examples, an example method for occluding an anatomical chamber includes introducing the into an interior of the anatomical chamber. The method may further include securing the occluding pouch to the wall of the anatomical chamber by engaging at least one retention member of the plug with the wall. The method may further include expanding the occluding pouch by introducing a volume of biogel into an interior of the occluding pouch.

This application claims the benefit of U.S. Provisional Application No. 63/499,014, filed 28 Apr. 2023, the entire contents of which is incorporated herein by reference.

### TECHNICAL FIELD

The present disclosure generally relates to left atrial appendage occlusion, and to assemblies for left atrial appendage occlusion.

### BACKGROUND

Atrial fibrillation may result in thrombus formation, which may cause embolic strokes. In nonvalvular atrial fibrillation (AF), thrombi typically occur in the left atrial appendage (LAA). The LAA is a pocket connected to the left atrium, which normally contracts with the left atrium.

The LAA may include features such as muscular ridges and folds formed on the interior surface of the LAA, which may promote blood pooling and stagnation within the LAA and, consequently, the formation of blood clots. Likewise, the relatively small size of the LAA may promote the formation of blood clots. Additionally, in AF the heart may beat rapidly and often with less force, which may not allow the LAA to adequately receive and expel fresh blood from within. If a blood clot escapes the LAA and enters the left atrium to be circulated throughout the body, there is a risk that the clot will cause an embolism and stroke. However, AF may cause the LAA to insufficiently contract, resulting in stagnant blood which may promote clot formation. Thrombi or clots released from the LAA may be associated with thromboembolic events.

LAA occlusion (LAAO) may be used to prevent thromboembolic events. For example, the LAA may be occluded to reduce or prevent formation of clots, or migration of clots from the LAA. Other procedures for preventing formation or migration of clots from the LAA include removal of the LAA, ligation or closure of the LAA, implanting a device within the LAA that prevents the flow of blood into and out of the LAA, and implanting a device within the LAA that functions as a filter to allow the flow of blood therethrough but prevent the movement of larger particles, such as clots, from exiting the LAA. Drug therapy may include administration of oral anticoagulants to reduce or prevent clot formation. Clinicians or patients may prefer an alternative or complementary therapeutic treatment.

### SUMMARY

The present disclosure describes example assemblies and techniques for occluding an anatomical chamber. The anatomical chamber may be a left atrial appendage of a patient.

An example assembly for occluding an anatomical chamber according to aspects of the present disclosure includes an occluding pouch and a plug. The occluding pouch is configured to expand by receiving a volume of biogel to occlude the anatomical chamber. The plug includes at least one retention member configured to secure the occluding pouch to a wall of the anatomical chamber.

An example method for occluding an anatomical chamber according to aspects of the present disclosure includes introducing an assembly comprising an occluding pouch and a plug into an interior of the anatomical chamber. The method may further include securing the occluding pouch to a wall of the anatomical chamber by engaging at least one retention member of the plug with the wall. The method may further include expanding the occluding pouch by introducing a volume of biogel into an interior of the occluding pouch.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1A is a cross-sectional view of an example assembly including an occluding pouch and a plug.

FIG. 1B is a side view of the plug of FIG. 1A.

FIG. 1C is a cross-sectional view of the assembly of FIG. 1A with the occluding pouch filled with a volume of biogel in an expanded configuration.

FIG. 1D is a side view of an example assembly in which a portion of the plug and the retention member is free of the occluding pouch.

FIG. 2 is a partial view of a patient's heart with the assembly of FIG. 1C implanted in the left atrial appendage.

FIG. 3 is a cross-sectional view of an example assembly including an occluding pouch, a plug, and a balloon.

FIG. 4 is a side view of an example plug including a helical retention member.

FIG. 5 is a partial cross-sectional view of a pouch wall of an example occluding pouch including a coating.

FIG. 6 is a partial cross-sectional view of a pouch wall of an example occluding pouch including a textured surface.

FIG. 7A is a partial cross-sectional view of a pouch wall of an example occluding pouch including a shark-skin surface.

FIG. 7B is a partial front view of the pouch wall of FIG. 7A.

FIG. 8 is a partial cross-sectional view of an implanting assembly including a catheter, the occluding assembly of FIG. 1A, and an elongated pusher.

FIG. 9A is a partial view of a first stage of a process for occluding a left atrial appendage using the assembly of FIG. 8.

FIG. 9B is a partial view of a second stage of the process of FIG. 9A with the catheter being partially withdrawn.

FIG. 9C is a partial view of a third stage of the process of FIG. 9A with the catheter and the elongated pusher being withdrawn and the occluding assembly secured to a wall of the anatomical chamber.

FIG. 9D is a partial view of a fourth stage of the process of FIG. 9A with the occluding pouch expanded.

FIG. 9E is a partial view of a fifth stage of the process of FIG. 9A with the occluding pouch expanded and in occlusive contact with the wall of the anatomical chamber.

FIG. 10 is a flowchart showing an example technique for occluding an anatomical chamber of a patient with an occluding assembly.

### DETAILED DESCRIPTION

In general, aspects of the present disclosure relate to left atrial appendage occlusion, and to assemblies for left atrial appendage occlusion (LAAO). Occluding the LAAO may reduce or prevent formation or migration of thrombi from LAAO, which in turn may reduce or prevent thromboembolic events.

The size, orientation, and shape of the LAAO may significantly vary from individual to individual. For example, the number of lobes in the LAAO may vary from 1 to 4 lobes. The overall morphology may also vary according to four general shapes, including the “chicken wing” morphology (central lobe bending back on itself and it may have secondary lobes), the “windsock” morphology (dominant primary lobe variations in location and number of secondary/tertiary lobes), the “cauliflower” morphology (short length and more complex internal characteristics, most prone to embolic events) and the “cactus” morphology (dominant central lobe and secondary lobes arising superiorly and inferiorly). There may be some overlap between these morphologies depending on the angle of imaging or evaluation. The ostium (opening) of the LAAO from the left atrium may also differ in shape, for example, including an oval, triangular, foot-like, water drop-like, or round peripheral shape.

Conventional occlusive devices may require precise sizing to an individual patient's anatomy. For sizing, a clinician may need significant preparation and imaging of the patient before an occlusion procedure. Further, the sizing determined by a clinician may be susceptible to the type of imaging, and the imaging angle that is used. Imaging may include transesophageal echocardiography (TEE) or intracardiac echocardiography (ICE). The clinician may also need to account for the size and shape of the ostium, and the presence of adjacent structures. Conventional devices may not be sufficiently adjustable to the configuration of an individual patient's LAAO, and may yet allow partial leakage of thrombi from the LAAO even after deployment. For example, such devices may be susceptible to peri-device leaks. Certain devices may also exhibit dislodgement and embolization of the device away from the LAAO. Other complications may include device-related thrombus, and pericardial effusion.

Assemblies and methods according to the disclosure provide treatment by LAAO that is responsive to the configuration of an individual patient's anatomy. For example, occlusion assemblies according to the present disclosure may accommodate various types of LAA anatomies, and may be used to permanently occlude the left atrial appendage to reduce or prevent thromboembolic events in patients who are suffering from atrial fibrillation (AF) or are at risk of stroke.

An example assembly for occluding an anatomical chamber according to aspects of the present disclosure includes an occluding pouch and a plug. The occluding pouch is configured to expand by receiving a volume of biogel to occlude the anatomical chamber.

The plug includes at least one retention member configured to secure the occluding pouch to a wall of the anatomical chamber.

The expandable occluding pouch can accommodate different anatomies, for example, by expanding at various positions about the pouch into contact with adjacent walls of the LAAO. The plug may secure the occluding pouch in place within the LAAO during and after expansion, reducing or preventing migration, movement, or re-orientation of the occluding pouch beyond an acceptable extent.

An example method for occluding an anatomical chamber according to aspects of the present disclosure includes introducing an assembly comprising an occluding pouch and a plug into an interior of the anatomical chamber. The method may further include securing the occluding pouch to a wall of the anatomical chamber by engaging at least one retention member of the plug with the wall. The method may further include expanding the occluding pouch by introducing a volume of biogel into an interior of the occluding pouch.

Using a biogel may promote proper fit and stability, and reduce or prevent the occurrence of peri-device leaks that create pathways for clot formation. Further, the biogel may initially be flowable, for example, a flowable liquid or gel, which may allow better conformance to the LAAO configuration. In some examples, the biogel may cure or harden, promoting the retention of the occluding pouch in a shape, size, and configuration that substantially remains conforms to the LAAO.

Thus, assemblies and methods according to the present disclosure may provide clinicians with a relatively simpler, more forgiving and/or adaptable procedure. Patients may benefit from a relatively higher quality of life after treatment, compared with conventional devices. For example, with advice of the clinician, patients may be weaned off anticoagulation regimen post-procedure relatively faster or with greater confidence than compared to conventional devices or methods.

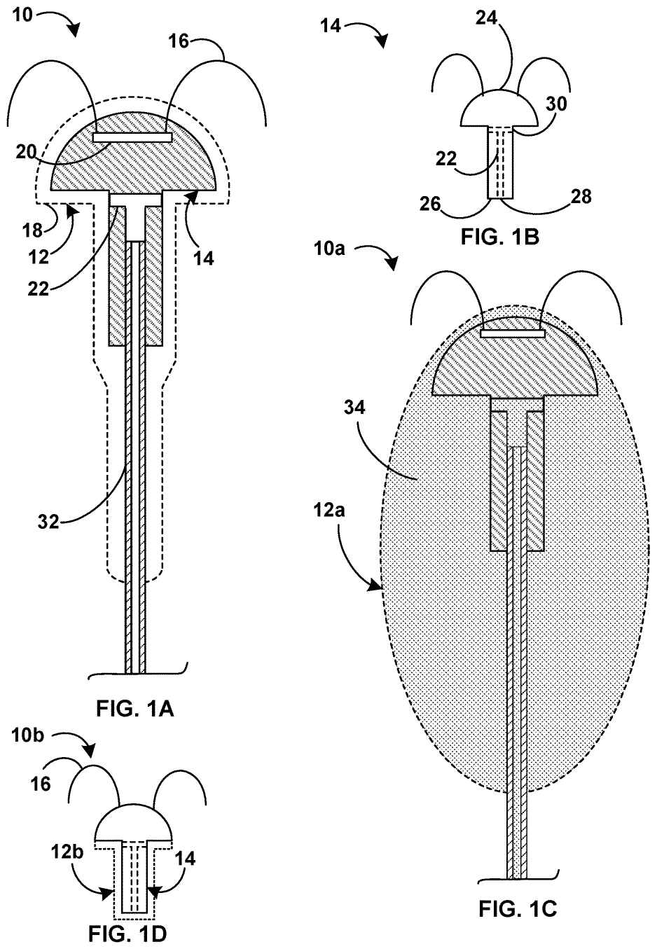

FIG. 1A is a cross-sectional view of an example assembly 10 including an occluding pouch 12 and a plug 14. The occluding pouch 12 is configured to expand by receiving a volume of biogel (shown in FIG. 1C) to occlude an anatomical chamber. Plug 14 includes at least one retention member 16 configured to secure occluding pouch 12 to a wall of the anatomical chamber. At least one retention member 16 may be configured to extend into a pouch wall 18 of occluding pouch 12 to engage the wall of the anatomical chamber. At least one retention member 16 may extend partially or completely through pouch wall 18. At least one retention member 16 may include at least one tine (as shown in FIGS. 1A to 1C) or at least one helical member (as shown in FIG. 4), as examples. At least one retention member 16 may be configured to exhibit a change in shape in response to contact or engagement with the wall of the anatomical chamber, or may be configured to substantially retain a shape without change. At least one retention member 16 may include a metal or an alloy.

At least one retention member 16 may be secured to plug 14 with an adhesive, a weld, an overmold, molding-in-place, or any other suitable technique or combinations thereof. In some examples, at least one retention member 16 is secured to a retention element 20, such as a plate or ring secured to plug 14. For example, retention element 20 may be embedded in or overmolded within plug 14. Retention element 20 may include a metal, an alloy, a plastic, a composite, or any suitable material or combinations thereof, and may be formed from a unitary piece of material with retention member(s) 16.

Plug 14 may be housed within occluding pouch 12. For example, pouch 12 may substantially surround an entirety of plug 14. Plug 14 may remain independently movable within pouch 12. Thus, while plug 14 may be constrained within pouch 12, plug 14 may yet have one or more degrees of freedom in an interior of pouch 12.

FIG. 1B is a side view of plug 14 of FIG. 1A. Plug 14 may have any suitable shape configured to facilitate entry of assembly 10 within the anatomical chamber and securement of the assembly 10 to the wall of the anatomical chamber. For example, one or more sections of plug 14 may be ellipsoidal, polyhedral, or have any solid shape. Plug 14 may be symmetric or asymmetric about a major axis. In some examples, plug 14 includes an elongated stem coupled to a rounded head, as shown in FIG. 1B. In some examples, plug 14 is mushroom-shaped, as shown in FIG. 1B.

Plug 14 may include a rigid material, for example, a material that promotes the retention of the shape and form of plug 14 during use of plug 14, and a material that resists deformation of plug 14. The rigid material may include at least one polymeric material. In some examples, the rigid material includes polyether ether ketone (PEEK). Plug 14 may include a radiopaque or echogenic marker.

While plug 14 includes retention member 16 in the example shown in FIGS. 1A to 1C, in other examples, plug 14 may not include retention member 16. For example, plug 14 may be retained in the anatomical chamber without engagement with a retention member. In some examples, one or both of pouch 12 or plug 14 are configured to facilitate retention of assembly 10 in the anatomical chamber. For example, a geometry (e.g., shape or size) or surface characteristic (e.g., roughness, texture, or coating) of one or both of pouch 12 or plug 14 may facilitate retention of assembly 10 in the anatomical chamber.

In some examples, plug 14 defines a biogel channel 22 configured to transport biogel into an interior of pouch 12. For example, plug 14 may include an elongated stem, and the elongated stem may define biogel channel 22. Plug 14 may extend between a distal plug end 24 configured to face the wall of the anatomical chamber and a proximal plug end 26 facing away from the wall. Biogel channel 22 may extend distally from a biogel inlet 28 defined by proximal end 26 to at least one biogel outlet 30. Biogel channel 22 may have a substantially constant cross-section along a portion of or along an entirety of biogel channel 22, or may include different segments with different cross-sections. Biogel channel 22 may include a segment having a continuously varying cross-section. Biogel channel 22 may have any suitable cross-sectional shape, including circular, elliptical, polygonal, or any other piecewise or continuous curved or linear contour segments.

In some examples, biogel channel 22 defines a T-shape extending from biogel inlet 28 to a pair of opposed biogel outlets 30. The pair of biogel outlets may be transverse to a plug axis extending between distal plug end 24 and proximal plug end 26.

Assembly 10 may further include an elongated cannula 32 configured to transport biogel to occluding pouch 12. For example, plug 14 may define biogel channel 22 configured to transport biogel into an interior of pouch 12, and elongated cannula 32 may be fluidically coupled to biogel channel 22. In some examples, a distal portion of elongated cannula 32 is positioned within a portion of biogel channel 22, such that a lumen of cannula 32 is fluidically coupled to biogel channel 32. Thus, biogel may be transported from an external source of biogel, such as a container including biogel, via cannula 32 (or otherwise) through biogel channel 22 and into interior of pouch 12 to cause pouch 12 to expand.

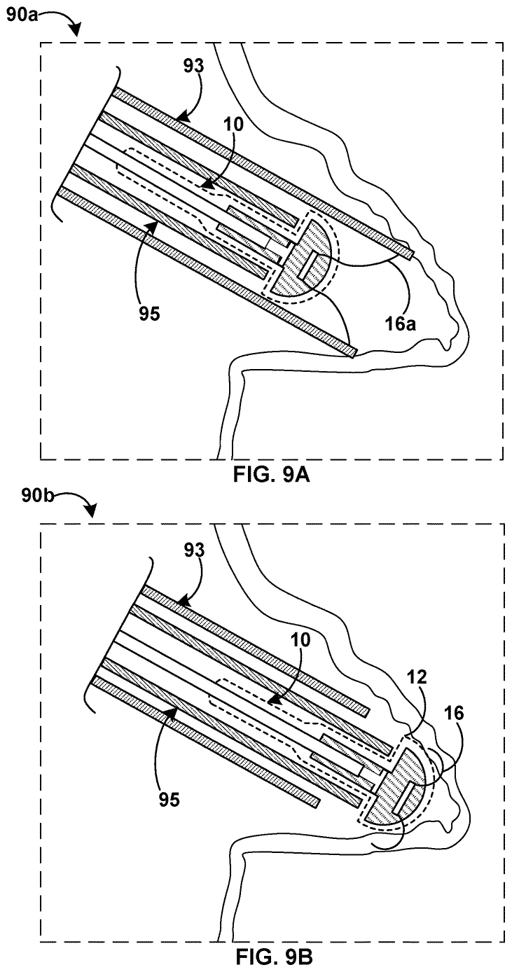

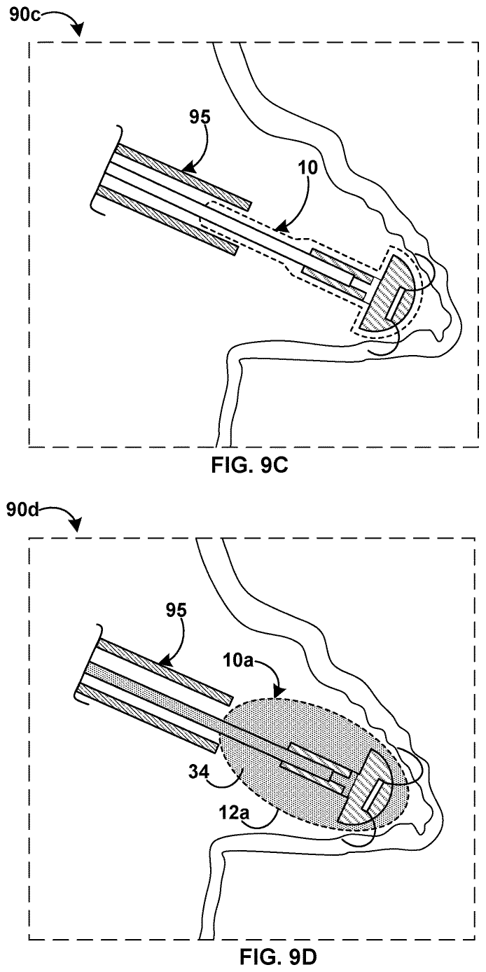

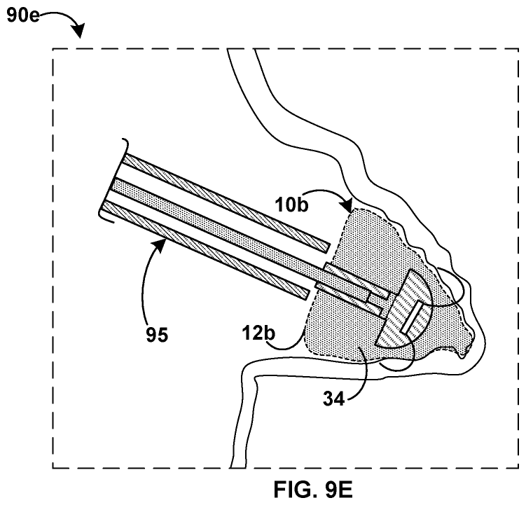

FIG. 1C is a cross-sectional view of assembly 10 of FIG. 1A with occluding pouch 12 filled with a volume of biogel 34 in an expanded configuration 10a. Expanded configuration 10a shown in FIG. 1C is an unconstrained configuration (not within an anatomical chamber and not externally constrained) for illustration. Thus, absent an external constraint, pouch 12 may expand from an initial shape to a fully expanded shape, as volume of biogel 34 within interior of pouch 12 increases. For example, occluding pouch 12 may be configured to expand from a first shape substantially conforming to plug 14 (as shown in FIG. 1A) to a second shape 12a (shown in FIG. 1C). If an external constraint is present, for example, an adjacent wall of an anatomical structure, second shape 12a may vary based on the constraint. For example, as described with reference to FIG. 9E, second shape 12a may substantially conform to at least a portion of the left atrial appendage. If expanded without constraint (such as a wall of an anatomical chamber), the second shape may be an ellipsoid, a polyhedron, a rectanguloid, a pyramid, or any other suitable shape or form.

Occluding pouch 12 may include a stretchable material or an elastic material. Pouch 12 may be reversibly or irreversibly deformable. For example, pouch 12 may be plastically and irreversibly deformable from a first (unexpanded shape) to a second (expanded shape). In other examples, pouch 12 may be reversibly expandable, for example, in response to changes in volume of biogel 34 (or another fluid or gas) within pouch 12. Occluding pouch 12 may include one or more metal, alloy, or polymeric materials, or combinations thereof. In some examples, pouch 12 includes a nylon or a polyester, or combinations thereof.

Pouch 12 may define a smoothly varying exterior or interior surface, or include segments or patches that are adhered, welded, molded or otherwise coupled together. Pouch 12 may be integral or unitary, or be formed of more than one discrete segment. Pouch 12 may include a single layer, or include a plurality of layers of material. Different layers may differ in construction or composition. One or more layers of pouch 12 may include a continuous film, a perforated film, a porous film, a woven material, or a non-woven material.

Pouch 12 may include a non-porous pouch wall 18. For example, pouch 12 may include a porous or meshed pouch wall 18 configured to retain the volume of biogel 34. The viscosity, gelation, or other fluidic properties of biogel 34 may cause biogel 34 to be retained within pouch 12 even if pouch wall 18 includes pores or a mesh.

While an entirety of plug 14 may be housed in occluding pouch 12, in other examples, a portion of plug 14, and at least one retention member 16, may not be housed in occluding pouch 12.

FIG. 1D is a side view of an example assembly 10b in which a portion of plug 14 and at least one retention member 16 is free of an occluding pouch 12b. Occluding pouch 12b is similar to occluding pouch 12 described with reference to FIG. 1A, but housing only a portion of plug 14. For example, a first portion of plug 14 may be housing in occluding pouch 12, and a second portion of plug 14 may not be housed in occluding pouch 12. Thus, plug 14 may not be completely housed within occluding pouch 12. Likewise, at least one retention member 16 may not be housed within occluding pouch 12. In some examples, a portion of plug 14 to which at least one retention member 16 is secured may not be housed within occluding pouch 12. In some examples, occluding pouch 12 may be secured to or about a stem or a proximal portion of plug 14, while a head or a distal portion of plug 14 may be free of pouch 12.

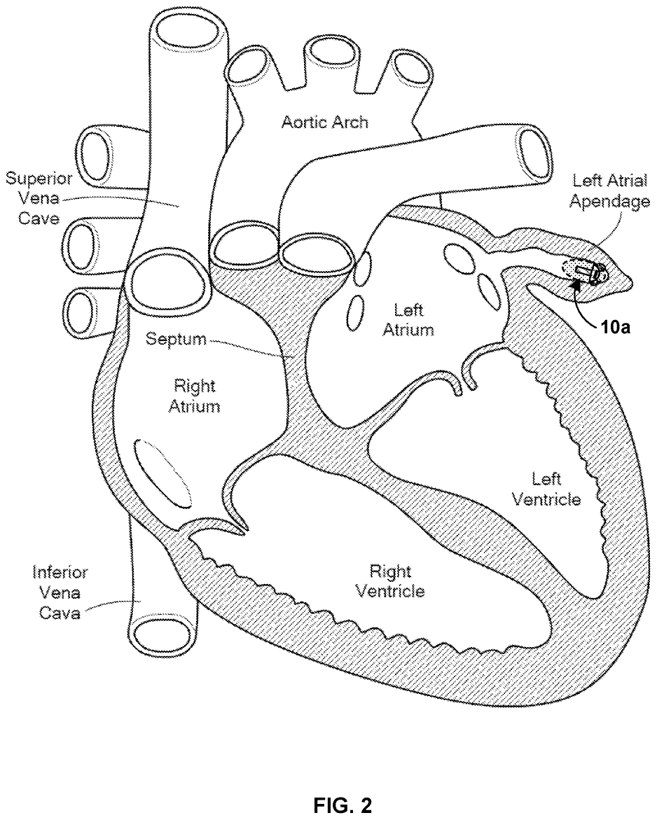

While assembly 10 may be used to occlude any anatomical structure, opening, or chamber, in some examples, assembly 10 may be used to occlude a left atrial appendage. Thus, the anatomical chamber described anywhere in the present disclosure may be a left atrial appendage of a patient, for example, as described with reference to FIG. 2.

FIG. 2 is a partial view of a patient's heart with assembly 10a of FIG. 1C implanted the left atrial appendage. In some examples, at least one retention member 16 is configured to engage an apex of the left atrial appendage.

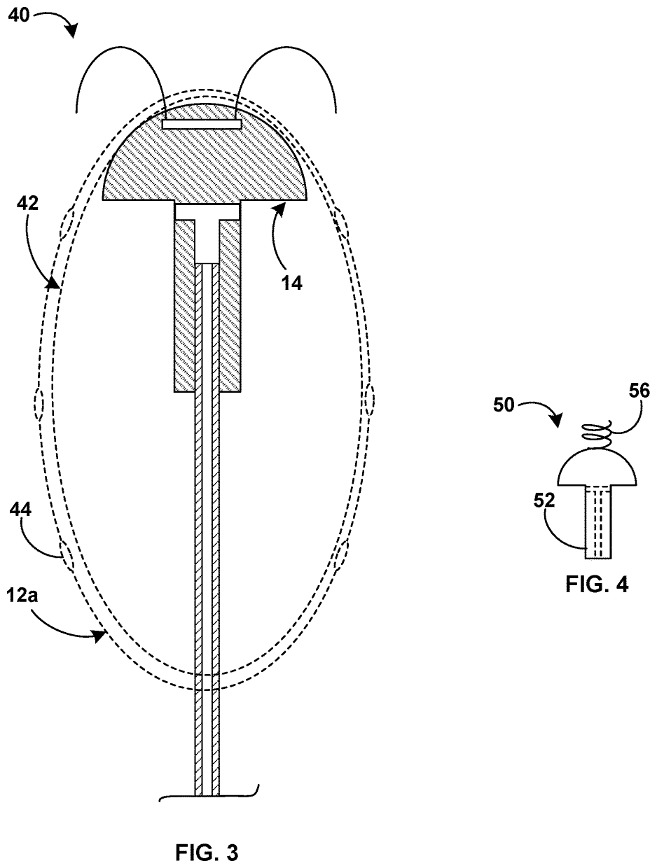

FIG. 3 is a cross-sectional view of an example assembly 40 including occluding pouch 12, plug 14, and a balloon 42, in an expanded configuration. Assembly 40 may be substantially similar to assembly 10 described with reference to FIGS. 1A to 1C, and differing in certain aspects as described herein. Balloon 42 may be positioned within occluding 12 pouch. Balloon 42 may be configured to reversibly inflate to cause occluding pouch 12 to reversibly expand in absence of the biogel. For example, as shown in FIG. 3, no biogel is present, and balloon 42 may be expanded with air (or some surgical, clinical, or sterile gas or gaseous mixture) or liquid (for example, a suitable surgical, clinical, or sterile liquid or solution such as saline).

Balloon 42 may be used to test expansion of pouch 12. For example, before pouch 12 is introduced within an anatomical chamber, balloon 42 may be inflated to facilitate checking or verifying the integrity of pouch 12 by the clinician. Alternatively, or in addition, balloon 42 may be inflated after assembly 10 is introduced in the anatomical chamber. For example, the clinician may position, reposition, orient, or reorient assembly 14 in various configurations in an anatomical chamber, and inflate balloon 42 to discern the likely expansion characteristics when pouch 12 is ultimately filled with biogel 34. After determining a suitable position, location, or orientation, the clinician may deflate balloon 42 and introduce biogel 34 within interior of pouch 12.

In some examples, instead of, or in addition to balloon 42, assembly 40 may include one or more additional pouches. For example, pouch 12 may be a first pouch, and assembly 40 may include second pouch 42 (balloon 42 may act as the second pouch). Likewise, the volume of biogel 34 may be a first volume of biogel, and second pouch 42 may receive a second volume of the same or a different biogel. While pouch 42 is within pouch 12 in FIG. 3, in other examples, the second pouch may surround the first pouch.

In some examples, pouch 12 include at least one imaging marker 44. At least one imaging marker may be a radiopaque marker or an echogenic marker.

While assembly member may include at least one tine as a retention member, in other examples, the at least one retention member may include other additional or alternative members, as described with reference to FIG. 4.

FIG. 4 is a side view of an example plug 50 including a plug body 52 and at least one helical retention member 56. Plug body 52 may be similar in construction and composition to plug 14 described with reference to FIGS. 1A to 1C. At least one helical retention member may include one or more coils or turns, and may facilitate securing of plug body 52 into a wall of an anatomical chamber. For example, an assembly including plug 50 may be screwed or turned to engage at least one helical retention member 56 with the wall. Providing at least one turn or coil may reduce or prevent inadvertent detachment of the assembly (whether assembly 10 or another assembly) including plug 50, for example, even if an axial pulling or pushing force is exerted on the assembly.

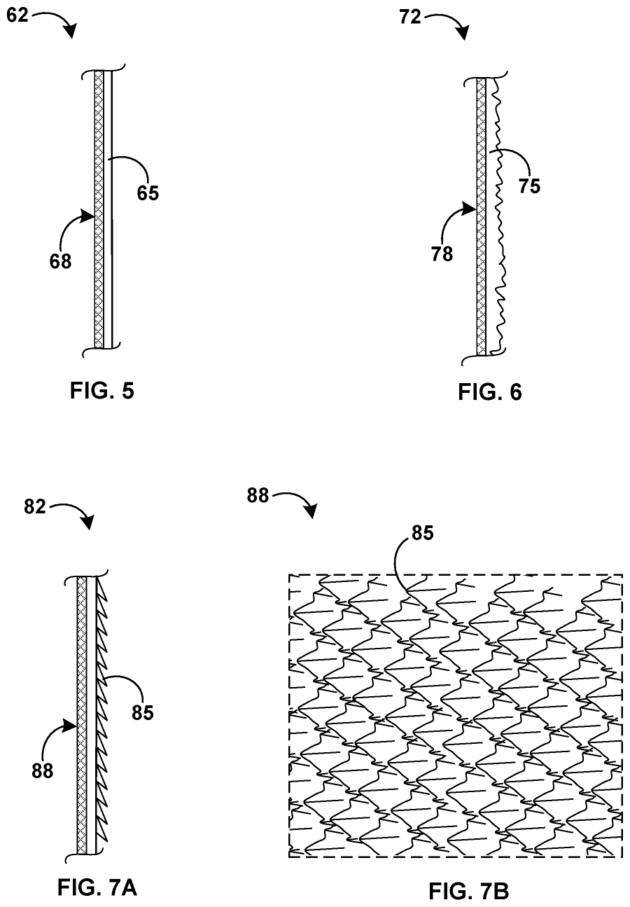

A pouch wall of a pouch may be coated, treated, or exhibit other features, as described with reference to FIGS. 5 to 7B.

FIG. 5 is a partial cross-sectional view of a pouch wall 68 of an example occluding pouch 62 including a coating 65. Pouch 62 and pouch wall 68 may be substantially similar in construction and composition to pouch 12 and pouch wall 68 described with reference to FIGS. 1A to 1C. Coating 65 may be a discrete coated disposed on a portion of or an entirety of pouch wall 68, or may constitute a layer of pouch wall 68. Coating 65 may include a biocompatible coating, for example, including a biocompatible polymer or other material, to reduce or prevent rejection of the assembly including pouch 62 by the patient's body. Coating 65 may reduce or prevent biofilm or clot formation. For example, coating 65 may include an antibiofouling material. In some examples, coating 65 may including a drug-eluting material.

FIG. 6 is a partial cross-sectional view of a pouch wall 78 of an example occluding pouch 72 including a textured surface 75. For example, pouch 72 may define textured surface 75 being configured to engage with the wall of the left atrial appendage. Textured surface 75 may be integral with pouch wall 78, for example, being defined by a surface or by a layer of pouch wall 78, or may constitute a discrete layer disposed on pouch wall 78. Textured surface 75 may include at least one of ridges, channels, stippling, pedestals, pits, teeth, serrations, friction pads, friction patterns, zig-zag members, raised features, embossed features, or any other suitable texture or combinations thereof.

FIG. 7A is a partial cross-sectional view of a pouch wall 88 of an example occluding pouch 82 including a shark-skin surface 85. FIG. 7B is a partial front view of pouch wall 88 of FIG. 7A. For example, a textured surface or shark-skin surface 85 may include or define a plurality of shark-skin members. The shark-skin members may be discrete members adhered, welded, molded or otherwise attached to pouch wall 88, or may be defined by a surface of pouch wall 88 or by a layer of pouch wall 88. In some examples, the shark-skin members comprise at least one polymeric material.

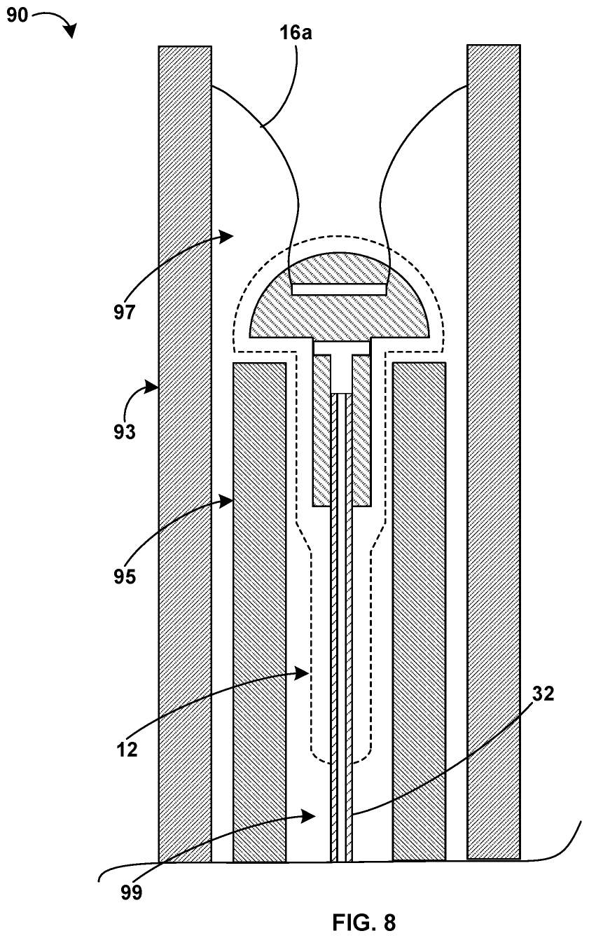

Assemblies described with reference to FIGS. 1A to 7A or assemblies including one or more components described with reference to FIGS. 1A to 7A may be used to occlude an anatomical chamber. In some examples, the anatomical chamber may be a left atrial appendage of a patient. Techniques and assemblies for occluding anatomical chambers are described with reference to FIGS. 8 to 10. While the techniques and assemblies of FIGS. 8 to 10 are described with reference to assembly 10 of FIGS. 1A to 1C, any assembly, device, or component according to the present disclosure may be used as suitable.

FIG. 8 is a partial cross-sectional view of an implanting assembly 90 including a catheter 93, occluding assembly 10 of FIG. 1A, and an elongated pusher 95. Occluding assembly 10 is positioned at a position along a lumen 97 of catheter 93. The upper end of catheter 93 corresponds to a distal end of catheter 93 facing a wall of an anatomical structure, such that occluding assembly 10 may be transported toward or away from the wall by axial movement along lumen 97. Elongated pusher 95 may be used to push occluding assembly 10 along lumen 97, for example, in a distal direction.

In the configuration shown in FIG. 8, at least one retention member 16 (at least one tine) is in a biased configuration 16a, ready to assume the engagement configuration 16 shown in FIG. 1A upon being released through a distal end of catheter 93. Thus, catheter 103 retains biased configuration 16a prior to engagement of plug 14 with the wall of the anatomical structure. Catheter 93 and elongated pusher 95 may include any suitable material, including at least one polymer, and may be reinforced with at least one reinforcing material or structure, for example, a metal or alloy reinforcing structure, such as coils, strands, fibers, particulates, rods, discs, or any other reinforcing structure.

Elongated pusher 95 may define a pusher lumen 99. A portion of assembly 10 may be received in pusher lumen 99. For example, a distal portion of pouch 12 and/or plug 14 may be received in pusher lumen 99. Cannula 32 may extend distally toward plug 14 through pusher lumen 99.

As described with reference to FIGS. 9A to 9E, catheter 93 and elongated pusher 95 may be operated independently to deploy assembly 90 in an anatomical chamber. The anatomical chamber may be a left atrial appendage.

FIG. 9A is a partial view of a first stage of a process for occluding a left atrial appendage using the implanting assembly 90 of FIG. 8 in a first configuration 90a. In the first configuration 90a, the at least one retention member is in biased configuration 16a, and elongated pusher 95 is ready to push assembly 10 to advance assembly 10 distally and beyond a distal end of catheter 93.

FIG. 9B is a partial view of a second stage of the process of FIG. 9A with catheter 93 being partially withdrawn proximally away from assembly 10 in a second configuration 90b, to release at least one retention member into engagement with the wall in configuration 16. The at least one retention member is now passing through assembly 10 into the wall, to securely retain assembly 10 in the anatomical chamber. Pusher 93 has been advanced distally compared to the first configuration 90a. Pusher 93 is now ready to be proximally retracted away from assembly 10, so that pouch 12 may be expanded.

FIG. 9C is a partial view of a third stage of the process of FIG. 9A with catheter 93 and elongated pusher 93 being withdrawn proximally, and occluding assembly 10 secured to the wall of the anatomical chamber (for example, left atrial appendage). Pouch 12 is now ready to receive volume of biogel 34 and to expand.

FIG. 9D is a partial view of a fourth stage of the process of FIG. 9A with the occluding pouch expanded in a fourth configuration 90d. The occluding pouch is now in expanded configuration 12a. In the fourth stage, occluding pouch 12a may or may not have contacted the wall of the anatomical chamber, and thus may or may not have at least partially conformed to a shape or an interior surface of the anatomical chamber. For example, as shown in FIG. 9D, expanded configuration 12a is yet ellipsoidal, and ready to conform to the anatomical chamber. Pusher 95 is ready to be again advanced distally toward the wall, but this time to push pouch 12 into substantially conforming contact with the wall, rather than to push assembly 10.

FIG. 9E is a partial view of a fifth stage of the process of FIG. 9A with occluding pouch expanded and in occlusive contact with the wall of the anatomical chamber in a fifth configuration 90e. In configuration 90e, the occluding pouch is in second expanded configuration 12b, being pushed or maneuvered by elongated pusher 95 into substantially conforming contact with the wall. At this stage, occluding pouch 12b can be considered as occluding the anatomical chamber.

However, in other examples, the occluding pouch may at least partially occlude the anatomical chamber even in the first expanded configuration 12a, for example, with partial or incomplete conformance with the wall of the anatomical chamber, or otherwise with sufficient occlusive contact with the wall of the anatomical chamber. Biogel 34 may not be cured or hardened, or otherwise sufficiently flowable, in between configurations 90d and 90e, to allow occluding pouch to transform from first expanded configuration 12a to second expanded configuration 12b. Biogel 34 may further be allowed to cure or harden, so that assembly 10 substantially remains in place, and resists embolization or migration away from the anatomical chamber.

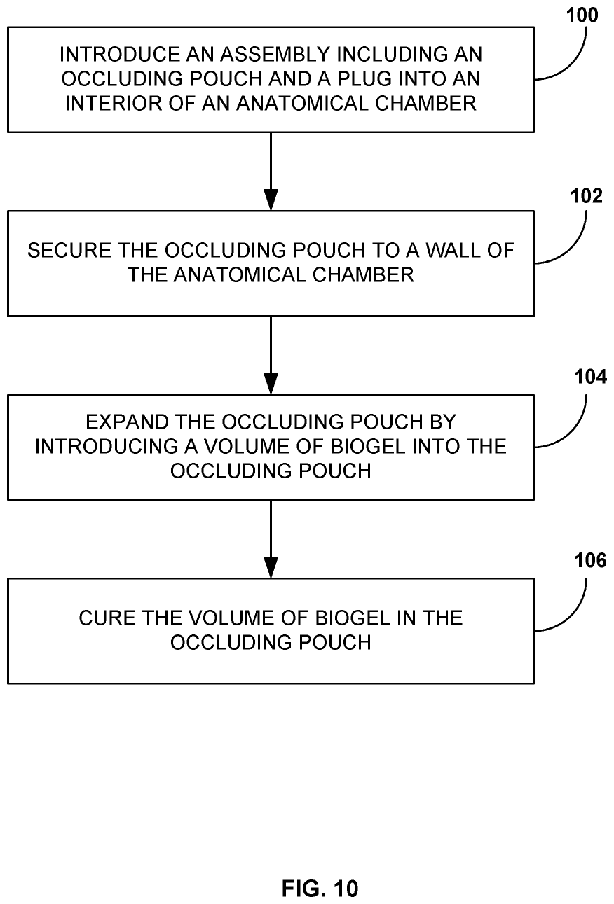

FIG. 10 is a flowchart showing an example technique for occluding a left atrial appendage of a patient with an occluding assembly.

An example method for occluding an anatomical chamber according to aspects of the present disclosure includes introducing an assembly including an occluding pouch and a plug into an interior of the anatomical chamber (100). The anatomical chamber may be a left atrial appendage of a patient.

The method may further include securing the occluding pouch to a wall of the anatomical chamber by engaging at least one retention member of the plug with the wall (102). Securing the occluding pouch (102) may include engaging the at least one retention member of the plug to an apex of the left atrial appendage. The at least one retention member may include at least one tine or at least one helical member, and engaging the at least one retention member (102) may include introducing a portion of the at least one tine or the at least one helical member through the wall of the anatomical chamber. In some such examples, introducing the portion of the at least one tine or the at least one helical member through the wall of the anatomical chamber includes passing the portion of the at least one tine or the at least one helical member through a pouch wall of the pouch.

The method may further include expanding the occluding pouch by introducing a volume of biogel into an interior of the occluding pouch (104).

The method of FIG. 10 may further include curing the volume of biogel in the occluding pouch. Curing the volume of biogel may increase the hardness of the volume of biogel in the occluding pouch within the anatomical chamber. In some examples, curing the volume of biogel includes introducing a curing agent into the pouch. Curing the volume of biogel may include auto crosslinking of the biogel, for example, crosslinking between components of the biogel without an external stimulus. Curing the volume of biogel may include thermal curing of the biogel. Thermal curing may include warming of the biogel from a first temperature lower than body temperature to a second temperature higher than the first temperature. For example, the biogel may be initially introduced into the occluding pouch at a temperature lower than body temperature, and warm to approach or reach body temperature in due course due to exposure to the anatomical chamber. Such warming may result in curing of the biogel over time.

In some examples, the volume of biogel includes a self-expanding material. For example, the biogel may include a foaming material that may tend to increase in volume to occupy any available volume as the occluding pouch expands. The biogel may include a radiopaque or echogenic agent.

In some examples, the volume of biogel is a first volume of biogel, and the method further includes introducing a second volume of biogel into the occluding pouch. In some such examples, the volume of biogel is a first volume of a first biogel, and the method further includes introducing a second volume of a second biogel into the occluding pouch.

The biogel may include at least one of a polyethylene, a polyurethane, a fibrin, a chitosan, a polyvinyl alcohol, an alginate, a cyanoacrylate, a silicone, a polyethylene glycol, or an acrylamide.

Introducing the volume of biogel into the interior of the occluding pouch may include transporting the volume of biogel through a biogel channel defined by the plug. The method may further include fluidically coupling an elongated cannula to the biogel channel, and introducing the volume of biogel through the elongated cannula.

Introducing the volume of biogel into the interior of the occluding pouch may cause the occluding pouch to expand from a first shape substantially conforming to the plug to a second shape substantially conforming to at least a portion of the left atrial appendage.

In some examples, the technique includes, before introducing the volume of biogel (104), inflating a balloon within the occluding pouch to reversibly expand the occluding pouch in absence of the biogel.

Introducing the assembly may include introducing a distal opening of a catheter into the left atrial appendage, and advancing the assembly distally within a lumen of a catheter through the distal opening into an interior of the left atrial appendage. Advancing the assembly distally within the lumen of the catheter may include pushing the assembly distally with an elongated pusher extending along the lumen of the catheter.

In some examples, the technique may include proximally retracting the catheter to allow the retention member to contact the wall of the left atrial appendage.

The technique may include with the elongated pusher, securing the assembly to the wall of the left atrial appendage by distally advancing the elongated pusher to push the retention member into engagement with the wall of the left atrial appendage. After securing the assembly to the wall, the elongated pusher may be retracted proximally away from the left atrial appendage. Retracting the elongated pusher may be performed prior to expanding the occluding pouch.

In some examples, after expanding the occluding pouch, the elongated pusher is advanced distally to promote occlusive contact and conforming of the occluding pouch with the wall of the left atrial appendage.

Enumerated examples according to the present disclosure are listed below.

Example 1: An assembly for occluding an anatomical chamber, the assembly including: an occluding pouch configured to expand by receiving a volume of biogel to occlude the anatomical chamber; and a plug including at least one retention member configured to secure the occluding pouch to a wall of the anatomical chamber.

Example 2: The assembly of example 1, where the anatomical chamber is a left atrial appendage of a patient.

Example 3: The assembly of example 2, where the at least one retention member is configured to engage an apex of the left atrial appendage.

Example 4: The assembly of example 1, where the plug is housed within the occluding pouch.

Example 5: The assembly of example 3, where the at least one retention member is configured to extend through a pouch wall of the pouch to engage the wall of the anatomical chamber.

Example 6: The assembly of any of examples 1 to 5, where the at least one retention member includes at least one tine or at least one helical member.

Example 7: The assembly of any of examples 1 to 6, where the at least one retention member includes a metal or an alloy.

Example 8: The assembly of any of examples 1 to 7, where the at least one retention member is secured to a retention element embedded in the plug.

Example 9: The assembly of any of examples 1 to 8, where the plug includes an elongated stem coupled to a rounded head.

Example 10: The assembly of example 9, where the plug is mushroom-shaped.

Example 11: The assembly of any of examples 1 to 10, where the plug includes a rigid material.

Example 12: The assembly of example 11, where the rigid material includes polyether ether ketone (PEEK).

Example 13: The assembly of any of examples 1 to 12, where the plug includes a radiopaque marker.

Example 14: The assembly of any of examples 1 to 13, where the plug defines a biogel channel configured to transport biogel into an interior of the pouch.

Example 15: The assembly of example 14, where the plug includes an elongated stem, and where the elongated stem defines the biogel channel.

Example 16: The assembly of examples 14 or 15, where the plug extends between a distal plug end configured to face the wall of the anatomical chamber and a proximal plug end facing away from the wall, and where the biogel channel extends distally from a biogel inlet defined by the proximal end to at least one biogel outlet.

Example 17: The assembly of example 16, where the biogel channel defines a T-shape extending from the biogel inlet to a pair of opposed biogel outlets.

Example 18: The assembly of example 17, where the two biogel outlets are transverse to a plug axis extending between the distal plug end and the proximal plug.

Example 19: The assembly of any one of examples 1 to 18, where the occluding pouch is configured to expand from a first shape substantially conforming to the plug to a second shape, where the second shape substantially conforms to at least a portion of the left atrial appendage.

Example 20: The assembly of any of examples 1 to 19, where the occluding pouch includes a stretchable material or an elastic material.

Example 21: The assembly of example 20, where the occluding pouch includes a nylon or a polyester.

Example 22: The assembly of any of examples 1 to 21, where the pouch includes a non-porous pouch wall.

Example 23: The assembly of any of examples 1 to 21, where the pouch includes a porous or meshed pouch wall configured to retain the biogel.

Example 24: The assembly of any of examples 1 to 23, where the pouch includes an antibiofouling coating.

Example 25: The assembly of any of examples 1 to 24, where the pouch defines a textured surface configured to engage with the wall of the left atrial appendage.

Example 26: The assembly of example 25, where the textured surface includes a plurality of shark-skin members.

Example 27: The assembly of any of examples 1 to 26, further including an elongated cannula configured to transport biogel to the occluding pouch.

Example 28: The assembly of example 27, where the plug defines a biogel channel configured to transport biogel into an interior of the pouch, and where the elongated cannula is configured to be fluidically coupled to the biogel channel.

Example 29: The assembly of any of examples 1 to 28, further including a balloon within the occluding pouch, where the balloon is configured to reversibly inflate to cause the occluding pouch to reversibly expand in absence of the biogel.

Example 30: The assembly of any of examples 1 to 29, where the pouch is a first pouch, where the volume of biogel is a first volume of biogel, and where the assembly further includes a second pouch surrounding the first pouch and configured to expand by receiving a second volume of biogel.

Example 31: A method for occluding an anatomical chamber, the method including: introducing an assembly including an occluding pouch and a plug into an interior of the anatomical chamber; securing the occluding pouch to a wall of the anatomical chamber by engaging at least one retention member of the plug with the wall; and expanding the occluding pouch by introducing a volume of biogel into an interior of the occluding pouch.

Example 32: The method of example 31, where the anatomical chamber is a left atrial appendage of a patient.

Example 33: The method of example 32, where securing the occluding pouch includes engaging the at least one retention member of the plug to an apex of the left atrial appendage.

Example 34: The method of any of examples 31 to 33, where the at least one retention member includes at least one tine or at least one helical member, and where engaging the at least one retention member includes introducing a portion of the at least one tine or the at least one helical member through the wall of the anatomical chamber.

Example 35: The method of example 34, where introducing the portion of the at least one tine or the at least one helical member through the wall of the anatomical chamber includes passing the portion of the at least one tine or the at least one helical member through a pouch wall of the pouch.

Example 36: The method of any of examples 31 to 35, further including curing the volume of biogel in the occluding pouch.

Example 37: The method of example 36, where curing the volume of biogel increases the hardness of the volume of biogel in the occluding pouch within the anatomical chamber.

Example 38: The method of examples 36 or 37, where curing the volume of biogel includes introducing a curing agent into the pouch.

Example 39: The method of examples 36 or 37, where curing the volume of biogel includes auto crosslinking of the biogel.

Example 40: The method of examples 36 or 37, where curing the volume of biogel includes thermal curing of the biogel.

Example 41: The method of example 40, where the thermal curing includes warming of the biogel from a first temperature lower than body temperature to a second temperature higher than the first temperature.

Example 42: The method of any of examples 31 to 41, where the volume of biogel includes a self-expanding material.

Example 43: The method of any of examples 31 to 42, where the biogel includes a radiopaque or echogenic agent.

Example 44: The method of any of examples 31 to 43, where the volume of biogel is a first volume of biogel, and where the method further includes introducing a second volume of biogel into the occluding pouch.

Example 45: The method of any of examples 31 to 44, where the volume of biogel is a first volume of a first biogel, and where the method further includes introducing a second volume of a second biogel into the occluding pouch.

Example 46: The method of any of examples 31 to 45, where the biogel includes at least one of a polyethylene, a polyurethane, a fibrin, a chitosan, a polyvinyl alcohol, an alginate, a cyanoacrylate, a silicone, a polyethylene glycol, or an acrylamide.

Example 47: The method of any of examples 31 to 46, where introducing the volume of biogel into the interior of the occluding pouch includes transporting the volume of biogel through a biogel channel defined by the plug.

Example 48: The method of example 47, further including fluidically coupling an elongated cannula to the biogel channel, and introducing the volume of biogel through the elongated cannula.

Example 49: The method of any of examples 31 to 48, where introducing the volume of biogel into the interior of the occluding pouch causes the occluding pouch to expand from a first shape substantially conforming to the plug to a second shape, where the second shape is substantially ellipsoidal.

Example 50: The method of any of examples 31 to 49, further including, before introducing the volume of biogel, inflating a balloon within the occluding pouch to reversibly expand the occluding pouch in absence of the biogel.

Example 51: The method of any of examples 31 to 50, where introducing the assembly includes introducing a distal opening of a catheter into the left atrial appendage, and advancing the assembly distally within a lumen of a catheter through the distal opening into an interior of the left atrial appendage.

Example 52: The method of example 51, where advancing the assembly distally within the lumen of the catheter includes pushing the assembly distally with an elongated pusher extending along the lumen of the catheter.

Example 53: The method of example 52, further including, proximally retracting the catheter to allow the retention member to contact the wall of the left atrial appendage.

Example 54: The method of example 53, further including, securing the assembly to the wall of the left atrial appendage by distally advancing the elongated pusher to push the retention member into engagement with the wall of the left atrial appendage.

Example 55: The method of example 54, further including, after securing the assembly to the wall, retracting the elongated pusher proximally away from the left atrial appendage.

Example 56: The method of example 55, where retracting the elongated pusher is performed prior to expanding the occluding pouch.

Example 57: The method of examples 55 or 56, further including, after expanding the occluding pouch, advancing the elongated pusher distally to promote occlusive contact and conforming of the occluding pouch with the wall of the left atrial appendage.

Various examples of the disclosure have been described. Any combination of the described systems, operations, or functions is contemplated. These and other examples are within the scope of the following claims.

1. An assembly for occluding an anatomical chamber, the assembly comprising:
an occluding pouch configured to expand by receiving a volume of biogel to occlude the anatomical chamber; and
a plug comprising at least one retention member configured to secure the occluding pouch to a wall of the anatomical chamber.

2. The assembly of claim 1, wherein the anatomical chamber is a left atrial appendage of a patient.

3. The assembly of claim 1, wherein the plug is housed within the occluding pouch.

4. The assembly of claim 3, wherein the at least one retention member is configured to extend through a pouch wall of the pouch to engage the wall of the anatomical chamber.

5-15. (canceled)

16. The assembly of claim 1, wherein the at least one retention member comprises at least one tine or at least one helical member.

17. The assembly of claim 1, wherein the at least one retention member comprises a metal or an alloy.

18. The assembly of claim 1, wherein the plug comprises an elongated stem coupled to a rounded head.

19. The assembly of claim 1, wherein the plug comprises a rigid material.

20. The assembly of claim 1, wherein the plug defines a biogel channel configured to transport biogel into an interior of the pouch.

21. The assembly of claim 1, wherein the occluding pouch is configured to expand from a first shape substantially conforming to the plug to a second shape, wherein the second shape substantially conforms to at least a portion of the left atrial appendage.

22. The assembly of claim 1, wherein the occluding pouch comprises a stretchable material or an elastic material.

23. The assembly of claim 1, further comprising a balloon within the occluding pouch, wherein the balloon is configured to reversibly inflate to cause the occluding pouch to reversibly expand in absence of the biogel.

24. A method for occluding an anatomical chamber, the method comprising:
introducing an assembly comprising an occluding pouch and a plug into an interior of the anatomical chamber;
securing the occluding pouch to a wall of the anatomical chamber by engaging at least one retention member of the plug with the wall; and
expanding the occluding pouch by introducing a volume of biogel into an interior of the occluding pouch.

25. The method of claim 24, wherein the anatomical chamber is a left atrial appendage of a patient.

26. The method of claim 24, wherein the at least one retention member comprises at least one tine or at least one helical member, and wherein engaging the at least one retention member comprises introducing a portion of the at least one tine or the at least one helical member through the wall of the anatomical chamber.

27. The method of claim 24, further comprising curing the volume of biogel in the occluding pouch.

28. The method of claims 24, wherein the volume of biogel comprises a self-expanding material.

29. The method of claim 24, wherein introducing the volume of biogel into the interior of the occluding pouch comprises transporting the volume of biogel through a biogel channel defined by the plug.

30. The method of claim 28, further comprising, before introducing the volume of biogel, inflating a balloon within the occluding pouch to reversibly expand the occluding pouch in absence of the biogel.

31. The method of claim 28, wherein introducing the assembly comprises introducing a distal opening of a catheter into the left atrial appendage, and advancing the assembly distally within a lumen of a catheter through the distal opening into an interior of the left atrial appendage.
